(19) United States
(12) Patent Application Publication
(10) Pub. No.: US 2006/0116589 A1
(43) Pub. Date: Jun. 1, 2006

(54) Weight scale having function of pulse rate meter or heartbeat rate meter

(75) Inventor: Won-Hee Park, Seoul (KR)

Correspondence Address: GREENBLUM & BERNSTEIN, P.L.C., 1950 ROLAND CLARKE PLACE, RESTON, VA, 20191, US

(73) Assignee: JAWON Medical Co., Ltd., Kyungsan-City (KR)

(21) Appl. No.: 11/272,748

(22) Filed: Nov. 15, 2005

(30) Foreign Application Priority Data
Nov. 22, 2004 (KR) 10-2004-0095729

(51) Int. Cl. A61B 5/02

(52) U.S. Cl. 600/500

(57) ABSTRACT

Disclosed is a weight scale having a function of a pulse rate meter or a heartbeat rate meter in which a weight, a pulse, and a heartbeat can be measured using a plus and minus electrode provided at a main part of the weight scale to catch athletic effects according to variation of the weight, the pulse, and the heartbeat measured before and after exercise of a subject, a function that brings the most appropriate exercise intensity can be provided to the subject using exercise intensity according to pulse and heartbeat, and stress of the subject is measured using heartbeat rate variability (HRV) representing regularity of pulse or heartbeat. The weight scale includes a plus and minus electrode collecting pulse or heartbeat data of a subject in contact with the sole of the foot or the palm of the hand of the subject, a body signal detector detecting a pulse signal or a heartbeat signal from the electrode, a load cell converting load of the subject into an electrical signal, an amplifier amplifying the signals measured by the body signal detector and the load cell, a memory storing data received from the amplifier, and a main operation processor operating and analyzing the data stored in the memory depending on a program previously provided, to output data on a pulse or heartbeat rate and a weight.

### BACKGROUND OF THE INVENTION

1. Field of the Invention

The present invention relates to a weight scale having a function of a pulse rate meter or a heartbeat rate meter, and more particularly, to a weight scale having a function of a pulse rate meter or a heartbeat rate meter in which a weight, a pulse, and a heartbeat can be measured using a plus and minus electrode provided at a main part of the weight scale to catch athletic effects according to variation of the weight, the pulse, and the heartbeat measured before and after exercise of a subject, a function that brings the most appropriate exercise intensity can be provided to the subject using exercise intensity according to pulse and heartbeat, and stress of the subject is measured using heartbeat rate variability (HRV) representing regularity of pulse or heartbeat.

2. Discussion of the Related Art

With increase of interest in health nowadays, various sports facilities are being developed. Of them, devices for measuring health are being developed.

A weight scale is a fundamental device of the devices for measuring health, and has been widely used in hospitals and currently manufactured for home use.

Constructions of a general weight scale and a pulse rate meter or a heartbeat rate meter are shown in FIG. 1 in detail. As shown in FIG. 1, the weight scale includes a load cell 1 for measuring a weight of a subject, an amplifier 2 amplifying weight data measured by the load cell 1, an operation processor 3 performing operation to measure the weight, a memory 4 storing the measured result, and a display 5 displaying the measured result. The pulse rate meter or the heartbeat rate meter includes a plus and minus electrode 11 necessarily required to sense a pulse signal or a heartbeat signal, an amplifier 12 amplifying the pulse signal or the heartbeat signal sensed by the electrode 11, an operation processor 13 operating the pulse or the heartbeat, a display 15 displaying the operated result, and a memory 14 storing the operated result. As another example, an infrared sensor may be used to measure the pulse or the heartbeat.

A pulse rate meter or a heartbeat rate meter using HRV for measuring the pulse or the heartbeat to identify stress is commercially sold. The pulse rate meter and the heartbeat rate meter are all required to prescribe accurate exercise. Also, the weight scale is fundamentally required to analyze body components and used to identify obesity of the subject by simply measuring a weight of the subject.

With well-being ages, interest in health increases, and effective exercise is required considering exercise prescription suitable for the subject and stress of the subject. It is necessary to identify stress related to the weight of the subject and exercise prescription related to the weight to effectively identify the body status of the subject. The weight scale and the pulse rate meter or the heartbeat rate meter are required for accurate exercise prescription. However, a problem occurs in that the weight scale and the pulse rate meter or the heartbeat rate meter are separately be used.

Furthermore, since the amplifier, the operation processor, the display, and the memory separately used have similar functions, a problem occurs in that these elements are doubly be used.

### SUMMARY OF THE INVENTION

The present invention is directed to a weight scale having a function of a pulse rate meter or a heartbeat rate meter, which substantially obviates one or more of the problems due to limitations and disadvantages of the related art.

An object of the present invention is to provide a weight scale having a function of a pulse rate meter or a heartbeat rate meter in which the function of the pulse rate meter or the heartbeat rate meter is carried out by additionally providing a plus and minus electrode, so that the cost can be reduced, and the pulse rate or the heartbeat rate is measured when the weight is measured, so that measurement time can be reduced, thereby improving reliability of measured data.

Additional features and advantages of the invention will be set forth in the description which follows, and in part will be apparent from the description, or may be learned by practice of the invention. The objectives and other advantages of the invention will be realized and attained by the scheme particularly pointed out in the written description and claims hereof as well as the appended drawings.

To achieve these and other advantages and in accordance with the purpose of the present invention, as embodied and broadly described, a weight scale having a function of a pulse rate meter or a heartbeat rate meter according to the present invention includes a plus and minus electrode collecting pulse or heartbeat data of a subject in contact with the sole of the foot or the palm of the hand of the subject, a body signal detector detecting a pulse signal or a heartbeat signal from the electrode, a load cell converting load of the subject into an electrical signal, an amplifier amplifying the signals measured by the body signal detector and the load cell, a memory storing data received from the amplifier, and a main operation processor operating and analyzing the data stored in the memory depending on a program previously provided, to output data on a pulse or heartbeat rate and a weight.

The electrode further has a function for collecting data to calculate body fat.

The weight scale may include an infrared sensor fixed to a main body part of the subject, such as toe and finger, instead of the electrode.

It is to be understood that both the foregoing general description and the following detailed description are exemplary and explanatory and are intended to provide further explanation of the invention as claimed.

### BRIEF DESCRIPTION OF THE ATTACHED DRAWINGS

The invention will be described in detail with reference to the following drawings in which like reference numerals refer to like elements wherein:

FIG. 1 is a block diagram illustrating a general weight scale;

FIG. 2A is a block diagram illustrating a weight scale having a function of a pulse rate meter or a heartbeat rate meter according to the present invention;

FIG. 2B illustrates an example of a weight scale having a function of a pulse rate meter or a heartbeat rate meter according to the present invention; and

FIG. 3A and FIG. 3B illustrate another example of a weight scale having a function of a pulse rate meter or a heartbeat rate meter according to the present invention, in which FIG. 3A is a block diagram illustrating another example of the weight scale and FIG. 3B is a perspective view illustrating the state that the weight scale is actually used.

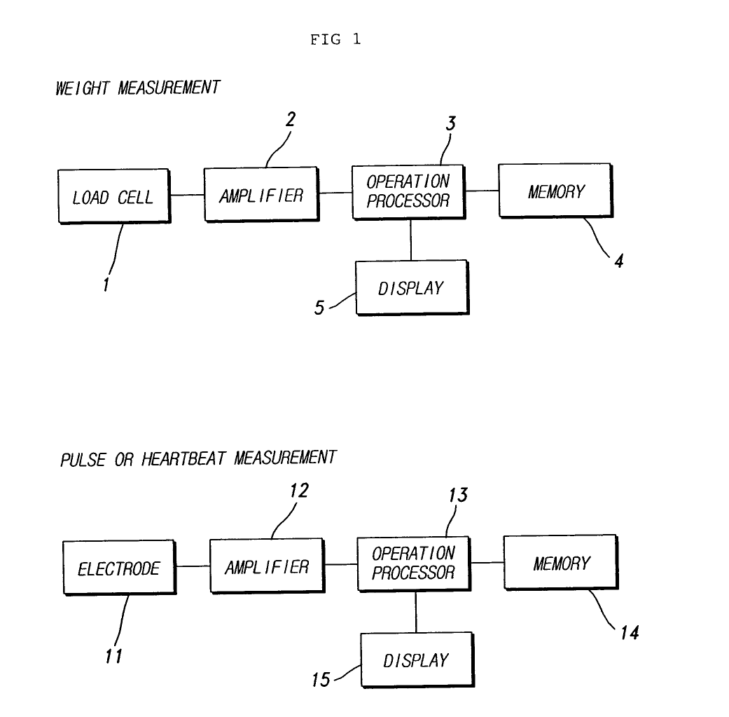

### DETAILED DESCRIPTION OF THE PREFERRED EMBODIMENTS

Reference will now be made in detail to the preferred embodiments of the present invention, examples of which are illustrated in the accompanying drawings.

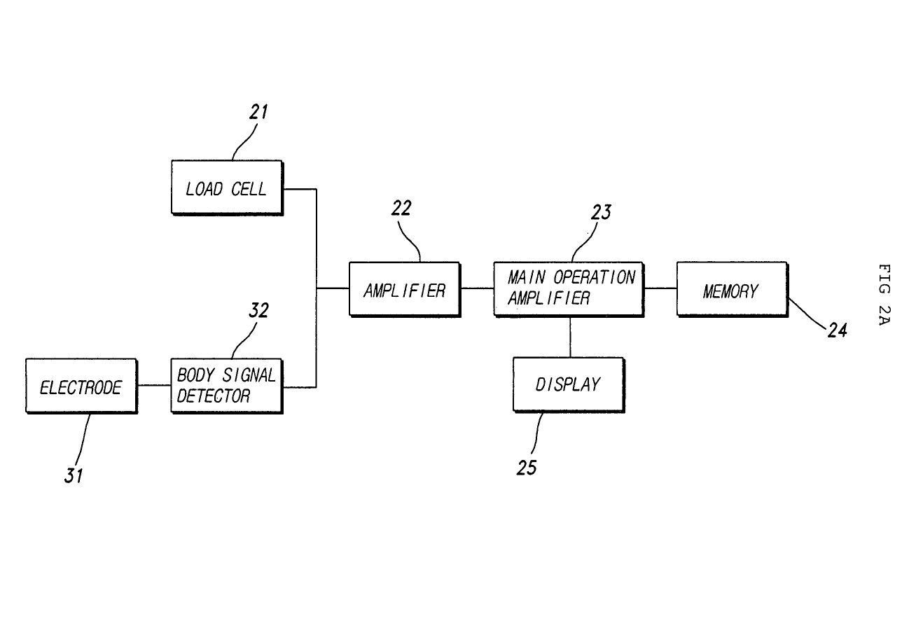

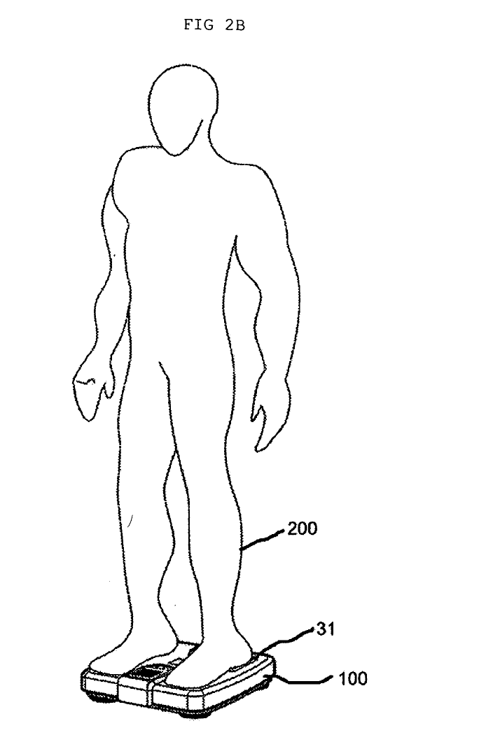

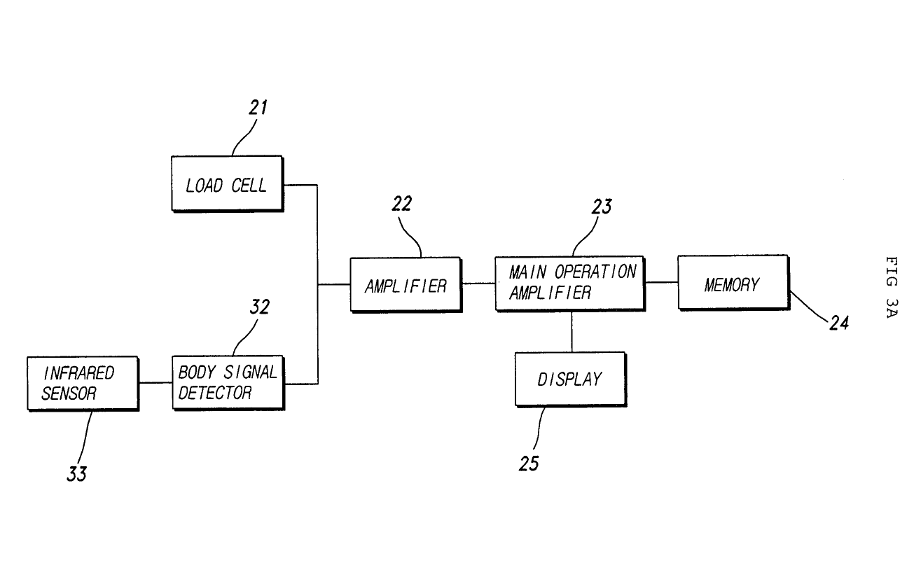

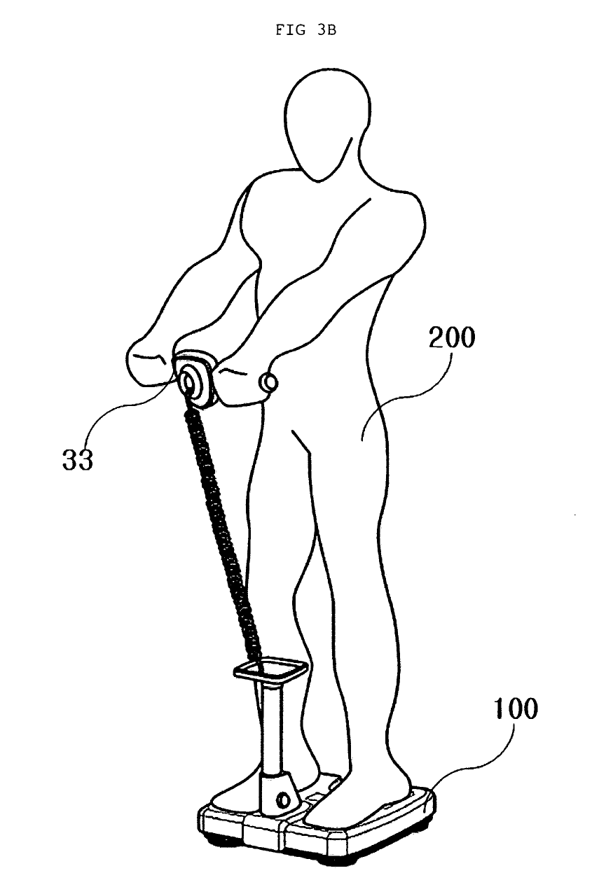

FIG. 2A is a block diagram illustrating a weight scale having a function of a pulse rate meter or a heartbeat rate meter according to the present invention, FIG. 2B illustrates an example of a weight scale having a function of a pulse rate meter or a heartbeat rate meter according to the present invention, and FIG. 3A and FIG. 3B illustrate another example of a weight scale having a function of a pulse rate meter or a heartbeat rate meter according to the present invention, in which FIG. 3A is a block diagram illustrating another example of the weight scale and FIG. 3B is a perspective view illustrating the state that the weight scale is actually used.

As shown in FIG. 2A and FIG. 2B, a weight scale of the present invention includes a plus and minus electrode 31 being in contact with the sole of the foot or the palm of the hand of a subject 200, a body signal detector 32 detecting a pulse signal or a heartbeat signal from the electrode 31, a load cell 21 converting load of the subject into an electrical signal, an amplifier 22 amplifying the pulse signal or the heartbeat signal detected by the body signal detector 32 and the electrical signal measured by the load cell 21, a memory 24 storing the signals received from the amplifier 22, a main operation processor 23 operating and analyzing the signals stored in the memory 24 depending on a program previously provided, and a display 25 displaying the signals processed by the main operation processor 23.

If the subject 200 stands on a weight scale 100 as shown in FIG. 2B, both soles of the feet are in contact with the electrode 31. In this state, measurement is carried out.

A weight and pulse or heartbeat rate data are measured by the load cell 21 and the body signal detector 32 and processed by the main operation processor 23. Then, the processed result is displayed on the display 25. Therefore, the weight and the pulse or heartbeat rate data before and after exercise are simultaneously collected to enable exact exercise prescription.

FIG. 3A and FIG. 3B illustrate another example of the weight scale having a function of a pulse rate meter or a heartbeat rate meter according to the present invention.

As shown in FIG. 3A, an electrode for measuring the pulse or heartbeat rate is used as an infrared sensor 33. In other words, another weight scale of the present invention includes a body signal detector 32 detecting a pulse signal or a heartbeat signal from the infrared sensor 33 fixed to a main body part of the subject 200, a load cell 21 converting load of the subject into an electrical signal, an amplifier 22 amplifying the pulse signal or the heartbeat signal detected by the body signal detector 32 and the electrical signal measured by the load cell 21, a memory 24 storing the signals received from the amplifier 22, a main operation processor 23 operating and analyzing the signals stored in the memory 24 depending on a program previously provided, and a display 25 displaying the signals processed by the main operation processor 23.

The subject 200 stands on the weight scale 100, as shown in FIG. 3B, to measure the weight. The pulse rate or heartbeat rate is measured through the infrared sensor 33 provided on the main body part of the subject. The weight and the pulse or heartbeat rate data are measured by the load cell 21 and the body signal detector 32 and processed by the main operation processor 23. Then, the processed result is displayed on the display 25. In the present invention, although the infrared sensor 33 is applied to the palm of the hand, it may be applied to fingers, toes, and the sole of the foot.

Therefore, the weight and the pulse or heartbeat rate data are measured before and after exercise of the subject in accordance with the present invention. In this way, the weight and the pulse or heartbeat rate data are simultaneously collected to enable exact exercise prescription. Also, variation of stress of the subject can be measured in accordance with the range of exercise.

The technology for measuring stress of the subject by measuring the pulse rate or the heartbeat rate is to measure stress of the subject by tracing an automatic nervous system (ANS) of the subject in a non-invasive method using heartbeat rate variability. The technology for measuring stress of the subject serves as an evaluating means to trace a homeostasis control mechanism of the automatic nervous system in response to internal and external environment factors. For example, a healthy person has great and complex heartbeat rate variability. However, it is noted that complexity of the heartbeat rate variability of the healthy person is remarkably reduced under the state of sickness or stress. The technology for measuring stress of the subject is developed as a pulse or heartbeat rate meter or an automatic nervous system meter.

Furthermore, for efficient exercise prescription, exercise intensity suitable for the subject should be determined. Also, it is important to calculate an exercise-consuming rate per shape of the subject depending on exercise intensity. To measure exercise intensity and exercise-consuming rate, it is essentially necessary to measure the weight and the pulse or heartbeat rate of the subject. American college of sports medicine (ACSM) recommends that minimum exercise intensity be prescribed depending on the weight and the pulse or heartbeat rate of the subject. Since the exercise intensity is calculated by consumed calorie per lkg, the weight of the subject is measured to calculate its consumed calorie. The pulse or heartbeat rate is used to determine the exercise intensity.

Since the pulse or heartbeat rate increases if the exercise intensity is strong, the pulse or heartbeat rate is measured after exercise to calculate how strong exercise has been done.

Therefore, the pulse or heartbeat rate and the weight of the subject are measured before and after exercise to determine efficient exercise intensity suitable for the subject. There has existed inconvenience in that both the pulse or heartbeat rate meter and the weight scale have been conventionally required. Also, it was difficult to simultaneously measure the pulse or heartbeat rate and the weight of the subject before and after exercise due to time difference between the case when measuring the weight and the case when measuring the pulse or heartbeat rate.

The weight scale having a function of a pulse rate meter or a heartbeat rate meter according to the present invention simultaneously measures the pulse or heartbeat rate and the weight before and after exercise to identify the body status of the subject. Thus, it is possible to measure exact exercise intensity of the subject and compare the pulse or heartbeat rate and the weight before exercise with them after exercise, thereby exactly measuring results according to exercise intensity. Further, since variation of stress of the subject before and after exercise can be measured by measuring heart rate variability of the pulse or heartbeat rate, the results of exercise can easily be evaluated. Since these procedures can be carried out by one measuring device, it is possible to reduce time and economical loss. Moreover, since the pulse or heartbeat rate and the weight are simultaneously measured, reliability of measured data increases.

As described above, the weight scale having a function of a pulse rate meter or a heartbeat rate meter according to the present invention has the following advantages.

The pulse or heartbeat rate and the weight can simultaneously be measured without additional device, and exercise intensity, variation of the weight, and the range of stress can be checked before and after exercise.

The foregoing embodiments are merely exemplary and are not to be construed as limiting the present invention. The present teachings can be readily applied to other types of apparatuses. The description of the present invention is intended to be illustrative, and not to limit the scope of the claims. Many alternatives, modifications, and variations will be apparent to those skilled in the art.

What is claimed is:

1. A weight scale having a function of a pulse rate meter or a heartbeat rate meter comprising:
a plus and minus electrode collecting pulse or heartbeat data of a subject in contact with the sole of the foot or the palm of the hand of the subject;
a body signal detector detecting a pulse signal or a heartbeat signal from the electrode;
a load cell converting load of the subject into an electrical signal;
an amplifier amplifying the signals measured by the body signal detector and the load cell;
a memory storing data received from the amplifier; and
a main operation processor operating and analyzing the data stored in the memory depending on a program previously provided, to output data on a pulse or heartbeat rate and a weight.

2. The weight scale according to claim 1, wherein the plus and minus electrode is provided on any one of the sole of the foot, the palm of the hand, and a finger.

3. A weight scale having a function of a pulse rate meter or a heartbeat rate meter comprising:
a plus and minus electrode collecting pulse or heartbeat data of a subject in contact with a body of the subject;
a body signal detector detecting a pulse signal or a heartbeat signal from the electrode;
a load cell converting load of the subject into an electrical signal;
an amplifier amplifying the signals measured by the body signal detector and the load cell;
a memory storing data received from the amplifier; and
a main operation processor operating the data stored in the memory depending on a program previously provided, to output data on a pulse or heartbeat rate and a weight,
wherein the weight and the pulse or heartbeat rate are simultaneously measured or the pulse or heartbeat rate is measured after the weight is measured and vice versa.

4. A weight scale having a function of a pulse rate meter or a heartbeat rate meter comprising:
an infrared sensor fixed to a main body part of a subject to collect pulse data of the subject;
a body signal detector detecting a pulse signal or a heartbeat signal from the sensor;
a load cell converting load of the subject into an electrical signal;
an amplifier amplifying the signals measured by the body signal detector and the load cell;
a memory storing data received from the amplifier; and
a main operation processor operating the data stored in the memory depending on a program previously provided, to output data on a pulse or heartbeat rate and a weight.

5. The weight scale according to claim 4, wherein infrared sensor is provided on any one of the sole of the foot, the palm of the hand, and a finger.

6. A weight scale having a function of a pulse rate meter or a heartbeat rate meter comprising:
an infrared sensor fixed to a main body part of a subject to collect pulse or heartbeat data of the subject;
a body signal detector detecting a pulse signal or a heartbeat signal from the sensor;
a load cell converting load of the subject into an electrical signal;
an amplifier amplifying the signals measured by the body signal detector and the load cell;
a memory storing data received from the amplifier; and
a main operation processor operating and analyzing the data stored in the memory depending on a program previously provided, to output data on a pulse or heartbeat rate and a weight,
wherein the weight and the pulse or heartbeat rate are simultaneously measured, or the pulse or heartbeat rate is measured after the weight is measured and vice versa.
